(19) United States
(12) Patent Application Publication
(10) Pub. No.: US 2019/0282376 A1
(43) Pub. Date: Sep. 19, 2019

(54) INSTRUMENT ASSEMBLY FOR USE IN HIP REPLACEMENT SURGERY

(71) Applicant: Depuy Ireland Unlimited Company, County Cork (IE)

(72) Inventors: Sarah Budenberg, Chesterfield (GB); Stephanie Prince, Wakefield (GB); Duncan Young, Cambridge (GB)

(21) Appl. No.: 16/320,539

(22) PCT Filed: Jul. 25, 2017
(86) PCT No.: PCT/GB2017/052172

§ 371 Date: Jan. 25, 2019

(30) Foreign Application Priority Data
Jul. 28, 2016 (GB) 1613058.5

(51) Int. Cl. A61F 2/46; A61B 90/00; A61B 17/17

(52) U.S. Cl. CPC A61F 2/4609; A61B 90/08; A61B 2090/0811; A61F 2002/4627; A61F 2002/4687; A61B 17/1746

(57) ABSTRACT

An instrument assembly and method for use in hip joint replacement surgery are described. The instrument assembly comprises a guide arm (102) having first (104) and second (106) ends, with a mount (108) at its first end (104) for locating the guide arm (102) relative to a landmark on the patient's back. An instrument (110) is provided at or towards the second end (106) of the guide arm (102) for modifying a patient's acetabulum. The guide arm (102) is arched to accommodate the patient's torso between its first and second ends (102, 104), and defines a reference axis extending between its first and second ends (102, 104) relative to which the instrument (110) is orientated. A surgical method is also described in which the instrument assembly is used in hip joint replacement surgery.

This invention relates to an instrument assembly for use in hip joint replacement surgery.

Commonly used hip joint prostheses include a cup component which is implanted in the patient's acetabulum. A two part cup component can comprise a shell which is implanted in the acetabulum and a liner which fits within the shell. The cup component (especially a liner within a shell) provides a concave articulation surface. A femoral component for implanting in the patient's femur has a stem which is fitted in the femoral intramedullary canal. A head is located superiorly and medially of the stem and has a convex articulation surface. The head is received in the cup component with the convex articulation surface of the head in contact with the concave articulation surface of the cup component.

It is important to ensure that the acetabular cup component is oriented accurately so that the joint has sufficient range of motion during articulation. The orientation of the acetabular cup component can be measured in terms of version and inclination angles. The version and inclination angles can be measured radiographically, typically with reference to the rim of the cup component. The inclination angle as defined in the radiographic reference system is the angle between the axis of the acetabular component and the patient's longitudinal axis, projected on to the coronal plane. The version angle in the radiographic reference system is the angle between the axis of the acetabular component and the coronal plane.

The version and inclination angles are also defined by convention in an anatomical reference system. The version angle is defined as the angle between the anatomic acetabular axis as projected on to the transverse plane and the transverse axis. The inclination angle is defined as the angle between the anatomic acetabular axis and the patient's longitudinal axis.

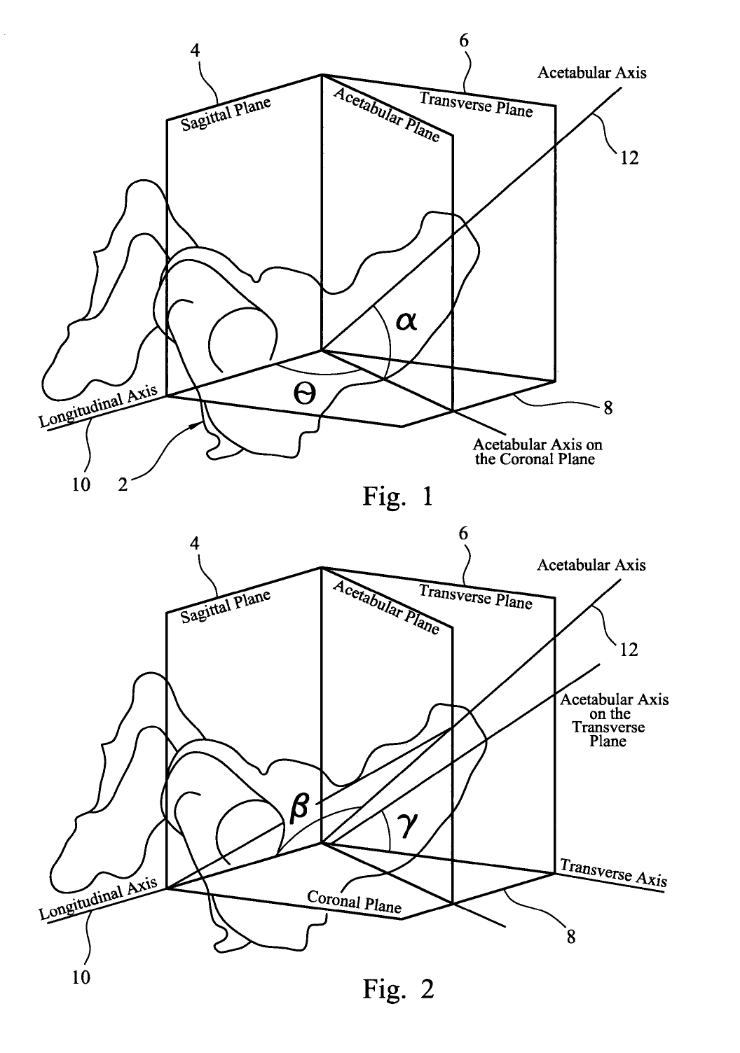

These features are shown in FIGS. 1 and 2, with FIG. 1 showing how the inclination and version angles are defined in the radiographic reference system (with the inclination angle labelled “θ” and the version angle labelled “α”) and FIG. 2 showing how the inclination and version angles are defined in the anatomical reference system (with the inclination angle labelled “β” and the version angle labelled “γ”. Typically, the version angle can be about 15 to about 20° and the inclination angle can be about 40 to about 45°, measured in the radiographic reference system. It can be the case that the version angle is in the range from about 15 to about 20° and the inclination angle is in the range from about 40 to about 45°, measured in the anatomical reference system.

It can be desirable to align an instrument which is used in a surgical procedure to implant a cup component in a patient's acetabulum. The instrument can be used to modify the acetabulum, for example in a step to prepare the acetabulum to receive a cup component, or to insert a cup component into the acetabulum. The instrument might be a cutting instrument which is used to cut bone or other tissue in or around the acetabulum. An example of an instrument which might be used to modify the acetabulum is a reamer which includes a cutting head having a part-spherical body with cutting teeth provided on its external surface. It can be used to modify the acetabulum by rotating it about its axis, causing the cutting teeth to act against the surface of the bone within the acetabulum. It can be desirable to align other cutting instruments which are used when modifying an acetabulum, such as for example a drill which drills holes in the acetabulum to receive screws or other fixation components to fix a cup component in place within the acetabulum. Another example of an instrument which can require appropriate alignment is a cutting guide, for example a guide for a drill which is used in a drilling step to form holes for screw or other fixation components, or a saw guide which is used to align a saw to cut bone.

It can be particularly desirable to align an inserter instrument which is used to position a cup component in a patient's acetabulum. The inserter instrument might be used to transmit an impaction force to a cup component to ensure that it is properly seated in the acetabulum. The cup component can be held in place within the acetabulum by being pressed into position. The cup component might then be retained in position in the acetabulum by means of fasteners such as screws. The cup component might be retained in position in the acetabulum by means of a bone cement. The surface of a cup component can have a porous region which can accommodate bone ingrowth after implantation to secure the cup component in place within the acetabulum.

An example of an instrument which can be used to align an acetabular cup component is disclosed in WO-A-2011/138327. The instrument includes a shaft with a mount at one end for a cup component. A guide arm is attached to the shaft. The guide arm is bent in two places so that it has first, second and third portions, in which the first portion is closest to the shaft and the third portion provides the free end of the guide arm. The first, second and third portions are co-planar. The angle between the first and second portions is about 40° and the angle between the second and third portion is about 90°. The shaft has a base fixed to it on which the guide arm is mounted. The base allows the orientation of the guide arm relative to the shaft to be adjusted. Use of the instrument involves positioning the arm relative to the shaft so that the shaft is aligned with the desired acetabular axis (which is the axis on which the acetabulum is to be modified, for example in a cutting step or in a step to implant a cup component) when the third portion of the arm is aligned with the plane of the operating table and with the patient's longitudinal axis.

Accurate use of the instrument which is disclosed in WO-A-2011/138327 relies on the patient's pelvis being positioned appropriately relative to the operating table and to the patient's longitudinal axis. It can therefore be preferred that, when the patient is in the lateral decubitus position, the spine and the pelvis are in their anatomical positions, that is with the spine in a plane which is parallel to the plane of the operating table, and the pelvis in a neutral position relative to the spine with zero degrees of displacement in each of flexion, abduction and rotation. Accurate use of the disclosed instrument also requires the surgeon to assess the alignment of the third portion of the guide arm with the plane of the operating table and the patient's longitudinal axis.

It can be challenging to control the position of a patient's pelvis accurately during hip replacement surgery. This can lead in some circumstances to inaccuracies in the positioning of an acetabular cup component.

The present invention provides an instrument assembly for use in hip joint replacement surgery which makes use of a guide arm having a first end which is located relative to a landmark on the patient's back, and an instrument at the second end of the arm which is a cutting tool or an inserter for an acetabular component, in which the guide arm defines an axis extending from its second end towards its first end relative to which the instrument is oriented.

The invention therefore provides an instrument assembly for use in hip joint replacement surgery, which comprises a guide arm having first and second ends, with a mount at its first end for locating the guide arm relative to a landmark on the patient's back, and an instrument at or towards the second end of the guide arm for modifying a patient's acetabulum, in which the guide arm is arched to accommodate the patient's torso between its first and second ends, and defines a reference axis extending between its first and second ends relative to which the instrument is orientated.

The assembly of the invention allows the instrument for modifying the acetabulum to be aligned relative to anatomical landmarks. This removes or at least reduces the need to control the position and orientation of the patient, especially of the patient's pelvis, during the surgical procedure in the way that is necessary when the instrument is aligned relative to other points of reference.

The guide arm will generally be shaped so that a portion between its first and second ends is offset relative to a straight line which extends between the ends. The guide arm will generally be shaped so that a portion between its first and second ends is offset relative to a straight line which extends between the first end of the guide arm and the point at or towards the second end of the guide arm at which the instrument is fastened to the guide arm. An offset portion of the guide arm can allow the guide arm to be fitted around the patient's torso with the first end of the guide arm positioned in contact with tissue on the patient's back, frequently close to the patient's spine, and the second end of the guide arm positioned above the patient's acetabulum so that the instrument is pointing towards the acetabulum, and generally towards the first end.

The guide arm can be provided by one or more members shaped as bars or rods or the like. Such members might have a solid cross section or they might be hollow along part or all of their lengths. However, the guide arm might be provided in other ways, for example by being cut from a sheet.

The instrument can be located with respect to the guide arm at or towards an end of the guide arm which is opposite to the end which is located on the patient's back. The instrument can be in contact with the guide arm. The instrument or the guide arm or each of them can be shaped in such a way as to restrict relative movement between the two when they are in contact with one another. The instrument can be connected to the guide arm. The connection might be one which allows the position (including the alignment) of the instrument relative to the guide arm to be changed. The connection might be a rigid connection which prevents relative movement between the instrument and the guide arm. It might be in contact with the guide arm at a position which spaced apart from the opposite end of the guide arm. For example, the guide arm might include an end portion which is located so that the instrument is in contact with the guide arm towards its second end, at a point along the length of the guide arm between its first and second ends. When the guide arm is shaped so that a portion between its first and second ends is offset, an end portion of the guide arm between the instrument and the second end can point in a direction from the instrument generally towards the first end. This can provide the user with indication of the orientation of the guide arm relative to features of the patient's anatomy, in particular relative to a selected landmark on the patient's back and to features on the patient's pelvis such as the patient's acetabulum.

The guide arm is arched so that the guide arm can accommodate the patient's torso, being located between the first and second ends so that the reference axis passes through the torso. The arch shape of the guide arm can be generally rounded. The arch shape can be provided by a crank-shaped portion. The arch shape can be made up of one or more straight portions. For example the arch shape might be made up of three straight portions which include two straight portions extending generally transversely (optionally perpendicularly) relative to the reference axis and a third straight portion between the two transverse straight portions which extends generally parallel to the reference axis. The guide arm can include can have an arcuate portion between the first and second ends defines a generally rounded arc, especially which is defined by a constant radius. The angle of arc defined by an arcuate portion of a guide arm can usefully be at least about 150°. The arcuate portion of the guide arm can extend between first and second straight portions of the guide arm which extend (or can be arranged to extend) along a line extending between the ends of the guide arm. The guide arm can have a generally crank-shaped portion which has a plurality of limbs.

Optionally, the guide arm can be telescopic. The effect that is available as a result of a guide arm being telescopic depends on the arrangement of the telescopic feature of the guide arm. For example, when the telescopic feature is provided in a portion of a guide arm which extends along or parallel to a line extending between the first and second ends of the guide arm, the feature will allow the length of the guide arm to be adjusted. When the telescopic feature is provided in a portion of the guide arm which is arcuate, especially with a constant radius, the feature will allow the angle between first and second end portions of the arm to be adjusted. This can be used to change the angular orientation of one of the end portions relative to the other end portion. When the first end of the guide arm has been located relative to a landmark on the patient's back and an instrument is fastened to the guide arm at its second end (especially in such a way that orientation of the instrument relative to the guide arm can be fixed), an adjustment of the telescoping portion of the guide arm can change the orientation of the instrument at the second end of the guide arm. This can be used to adjust the angle of the axis on which the acetabulum is modified using the instrument, for example to take account of factors such as particular anatomical features of the patient, or surgeon preference. This axis is referred to in this document as the “instrument axis”. It will extend through the acetabulum when the instrument is in use. Selection of the orientation of the instrument axis, extending from the acetabulum is facilitated by use of the guide arm.

The instrument assembly of the invention has the advantage that the guide arm provides the surgeon with a reference axis which is defined by the guide arm. An instrument used to modify a patient's acetabulum can be aligned with or with reference to this reference axis. In some circumstances, it might be preferred to align the instrument with an offset relative to the reference axis. Use of the assembly can reduce or eliminate the need for accurate control over the position or orientation of the patient's pelvis relative to a reference plane or axis because the pelvis itself can be used as a reference. Use of the assembly can also reduce or eliminate the need for the surgeon to estimate alignment of an instrument or part thereof relative to a reference plane or a reference axis which is spaced apart from the instrument.

When the assembly is in use, it can be preferred that the reference axis which is defined by the guide arm extends through the acetabulum. When the reference axis is angularly offset relative to the instrument axis, it can be preferred that the axes intersect at about the bony surface within the acetabulum.

The invention involves identification of a reference point on the patient's back. The reference point can be identified during a pre-operative planning step. It might be marked so that it can be located visually during the surgical procedure. For example, it might be marked using an ink marker.

The guide arm can have a mount at its first end which can located on skin on the patient's back relative to the reference point. For example, the mount can be fastened to the skin on the patient's back so that the first end of the arm is at the reference point. The reference point can be on the axis on which an acetabular cup component is to be aligned. The reference point can be displaced from the axis on which an acetabular cup component is to be aligned by a pre-determined distance or angle. The reference point is preferably such that it can be located by a procedure which includes manual examination (or palpation) of the patient. The reference point can be located with reference to bony landmarks which are capable of being located by manual examination. Examples of bony landmarks which can be identified readily in most patients include the posterior superior iliac spines (or PSIS points), features on the thoracic spine or the lumbar spine, the superior iliac spine, the sacrum and the coccyx. Examples of particularly useful landmarks on the patient's back can include one or other of the PSIS points, and the midpoint between the PSIS points. These landmarks can be located readily in most patients. For example, these landmarks can be located before surgery when a patient is sat on the operating table and can bend over to expose the lumbar spine. A landmark that is located in this way can be useful when locating a reference position for the desired acetabular axis (which is the axis extending through the acetabulum on which the acetabular cup component might ideally be aligned). The desired acetabular axis might extend through a reference point which lies at a landmark on the patient's back. The reference point might be spaced apart from a landmark. For example, the reference point might be midway between the PSIS, or spaced apart in a direction along the patient's superior-inferior axis from a landmark. It will often be preferred that the reference point is central on the patient's back, such that it lies on the patient's superior-inferior axis, or such that it lies on the sagittal plane which passes through the centerline of the patient's spinal column. The position of reference point relative to a landmark might be estimated, for example with reference to pre-operative images of the patient, or with reference to statistical anatomical data, or with reference to both images and anatomical data.

The identification of the location of the reference point can be performed during a pre-operative planning step. It can be performed with reference to images of the patient's pelvis, especially X-ray images.

Estimating the location of the reference point can be performed using statistical data relating the required displacement of the reference point from a landmark to dimensions of the patient's pelvis. For example, the location of the reference point can be related to the size of the pelvis measured parallel to the anterior-posterior axis or to the size of the pelvis measured parallel to the superior-inferior axis or to both of these measurements. The size of the pelvis measured parallel to the anterior-posterior axis can be measured between one of the posterior superior iliac spines and one of the anterior superior iliac spines. The size of the pelvis measured parallel to the superior-inferior axis can be measured between one of the posterior superior iliac spines and ischial tuberosity. The orientation of an acetabular component within the patient's acetabulum is characterised by inclination and anteversion angles. Appropriate inclination and anteversion angles for use when implanting an acetabular component in a patient might be estimated depending on the design of the component which is to be implanted in a patient, the steps in the procedure which the surgeon intends to use and, in some cases, the angle of the patient's pelvis when standing and when lying. In some cases, appropriate inclination and anteversion angles can also take account of factors such as the patient's sex and ethnicity, and the size of the patient's pelvis.

The location of the reference point can be adjusted for the purpose of selecting the angle of inclination (which is the angle between the axis of the acetabular component and the patient's longitudinal axis, projected on to the coronal plane) by selecting an appropriate displacement of the first end of the guide arm along the inferior-superior axis from the reference point. For example, in the case of a typical male patient having a height of about 180 cm and 17.5 cm from acetabular centre to acetabular centre, a displacement of 1 cm, in the inferior direction relative to a reference point located midway between the PSIS points, results in an increase in the inclination angle of about 2.3°.

The use of landmarks on the patient's back to locate the reference point has the advantage that the reference point might then be in a region where the quantity of soft tissue between the skin and underlying bone is small. In particular, it is envisaged that a reference point can be located on the skin in the region of the sacrum. The quantity of fat between the skin and the sacrum is relatively small, even in patients who are overweight. The sacrum can provide a stable firm surface for location of a mount at the reference point.

A mount can be provided at the first end of the guide arm and intended to be held in place manually relative to the reference point on the patient's back. The mount can be provided by the end surface of the guide arm. The mount can be provided by a part which is separate from the guide arm. The guide arm and a separate mount can be connected to one another. The guide arm and a separate mount can be shaped so that they can be placed in contact with one another without necessarily being connected to one another in such a way that resists separation. The mount can be shaped to facilitate it being gripped by a member of the surgical team, especially when the guide arm is able to move relative to the mount. The guide arm can be used to hold the mount in place, especially when the guide arm is fixed to the mount.

The mount can be positioned in place on the patient's back outside of a surgical drape or gown or other cover which lies over the patient. The cover can be transparent, at least around the reference point, to enable the person holding the guide to see the reference point. As mentioned above, the reference point might be marked after being identified during a pre-operative planning step to enable it then to be located during the surgical procedure.

When the mount is held against the patient's skin (or a drape or other cover which lies over the patient's skin), the mount can be shaped so that it sits stably against the patient's skin on the patient's back. For example, it might have an approximately flat pad which can sit against the skin. The connection between the mount and the first end of the guide arm can be rigid. The connection between the mount and the first end of the guide arm can allow the mount to articulate relative to the guide arm.

Optionally, the mount includes a quantity of adhesive on a surface which contacts the patient's skin when the mount is in place. It can be desirable for the adhesive sometimes to be breathable. It should have low potential for an allergenic reaction and should be capable of releasing from the skin surface with minimal likelihood of damaging the skin. Types of adhesives which can be used to fix the mount to skin include materials based on acrylic and silicone polymers, hydrocolloids, hydrogels, rubber-based materials, polyurethanes, polyesters and polyethers. Factors which might be taken into when selecting an appropriate adhesive material can include tackiness, ease of removal, moisture vapour transmission rate, compatibility with anti-microbial additives, and risk of allergic reaction. Acrylic based polymers can be particularly preferred because of their low cost, good tackiness, good peel strength, and good cohesive strength. Suitable materials are known from their use in wound care dressing applications.

A quantity of an adhesive can be provided on the mount as supplied for use in a surgical procedure. It might be appropriate for some procedures for the mount (and other components of the assembly) to be provided in a sterile condition with appropriate packaging to preserve the sterility. The adhesive can have a protective film applied to it which can be peeled off to expose the surface of the adhesive for use.

The assembly can provide a connector for connecting the guide arm to the mount. Optionally, the guide arm can have an arm connector formation at its first end and the mount can have a mount connector formation which can engage the arm connector formation to restrict movement of the arm relative to the mount. Optionally, the guide arm can have an arm magnetic connector part and the mount can have a mount magnetic connector part which give rise to an attraction force between the arm and the mount to hamper separation. Features of an instrument assembly having these feature are disclosed in a UK patent application which is being filed with this application with agents' reference SJB/P213137. Subject matter disclosed in that application is incorporated in this application by this reference. The mount can be fixed relative to an anatomical reference point on the patient's body in a preparatory step in a surgical procedure. The guide arm can then be engaged with the mount in a subsequent step, frequently at or just before the time at which an instrument which is engaged by the guide arm is used in the surgical procedure. The magnetic connector parts can be used to ensure alignment and subsequent engagement of the connector formations on the guide arm and the mount, facilitating alignment and connection even when the mount is not readily visible to the surgeon or other user.

A connector for connecting the guide arm to a separate mount can provide a plurality of fixation points for the first end of the guide arm. For example, a connector can provide at least one linear array of fixation points for the first end of the guide arm. An array of fixation points gives a surgeon the possibility of selecting a fixation point so that the first end of the guide arm is located appropriately relative to the reference point. An array of fixation points which is arranged along the patient's longitudinal axis give the surgeon the possibility of selecting the inclination angle of the desired acetabular axis to suit the requirements of a particular patient, as discussed above.

The connector and the guide arm can include spigot and socket features with one of the spigot and the socket being provided by the connector and the other of the spigot and the socket being provided by the guide arm. For example, the connector can provide one or more sockets and the guide arm can provide a spigot which is a sliding fit in the socket (or a selected one of the sockets). The spigot can fit in the socket in such a way that it is not capable of moving relative to the socket other than in a direction which is aligned with the insertion direction. The engagement of the spigot in the socket can therefore define an angular orientation of the guide arm relative to the connector.

The connector and guide arm can include ball and socket connector features with one of the ball and the socket being provided by the connector and the other of the ball and the socket being provided by the guide arm. The ball can be received in the socket to connect the guide arm to the connector. The ball can be capable of articulation within the socket.

A connector component which provides an array of fixation points can be provided as a part of a mount which is fastened to skin on the patient's back. A connector component which provides an array of fixation points can be provided as a part of a guide arm. A connector component which provides an array of fixation points can be provided as a part which is separate from each of the mount and the guide arm and can be connected to each of them. A connector component which is separate from the mount can be connected to the mount by means of connector formations and magnetic connector components. A connector component which is separate from the guide arm can be connected to the guide arm by means of spigot and socket features.

When the guide arm is shaped so that a portion between its first and second ends is offset relative to a straight line which extends between the ends, it can be preferred that portions of the guide arm along its length between its first and second ends lie in a common plane. When the guide arm has an arcuate portion between first and second end portions, the arcuate and first and second end portions can lie in a common plane. When the guide arm has a crank-shaped portion (made up of a plurality of limbs) positioned between first and second end portions of the guide arm, the crank-shaped portion and the first and second end portions can lie in a common plane.

The surface of a mount which is intended to be fitted against the patient's back will be configured to fit against the selected surface area of the patient's back. When the mount is to be fitted against the sacrum, the surface of the mount will frequently be essentially planar.

The use of a guide arm which includes a telescopic arcuate portion has the advantage that the orientation of the axis on which the acetabulum is modified using the instrument (the instrument axis) relative to the reference point at the first end of the guide arm can be adjusted by appropriately adjusting the length of the telescopic arcuate portion guide arm. The effect of adjusting the length of the guide arm on the version and inclination angles depends on the orientation of the guide arm (in particular of the plane which is defined by the guide arm when portions of the guide arm along its length between its first and second ends lie in a common plane) relative to the patient's longitudinal axis and to the coronal plane. It can be preferred that the plane defined by the guide arm is arranged so that telescopic adjustment of the length of the guide arm has little or no effect on the inclination angle so that adjustment of the length changes only the version angle (which is the angle between the axis of the acetabular component and the coronal plane). It can be preferred that the guide arm is arranged so that (i) the angle between the plane containing the guide arm and the anterior-posterior axis is small, for example less than 20°, preferably less than 10°, especially is 0±5°, or (ii) the angle between the plane defined by the arcuate portion of the guide arm and the superior-inferior axis is close to the angle of inclination, for example about 45°.

The guide arm might be constructed so that the angle between a line drawn perpendicular to the surface of the mount which is intended to be fitted against the patient's back and the plane which is defined by the portion of the guide arm which extends around the patient's torso is not more than about 20°, more preferably not more than about 10°, especially not more than about 5°. This would mean that, when the surface of the mount is parallel to the coronal plane, the plane which is defined by the guide arm is approximately perpendicular to the coronal plane when the guide arm is in use.

Optionally, the portion of the guide arm which extends around the patient's torso is approximately planar. This can facilitate control over the effect of an offset (which could be an angular offset or a displacement or a combination of the two) of the instrument axis relative to the reference axis such as for example when a fitting at the second end of the guide arm allows an offset of the instrument axis relative to the guide axis to be introduced. The effect of the offset can be controlled by aligning appropriately the plane defined by the portion of the guide arm which extends around the patient's torso.

The instrument assembly can include at least one alignment indicator which can be aligned with or with reference to an anatomical landmark. Optionally, at least one alignment indicator can be provided on the instrument which is used to modify the patient's acetabulum. The instrument assembly can be rotated about the instrument axis to align the instrument assembly angularly relative to the acetabulum. For example the alignment indicator can be positioned so that the instrument assembly is appropriately positioned rotationally when the alignment indicator is aligned with or with reference to the patient's anterior superior iliac spine. Two alignment indicators can be provided on the instrument to allow the assembly to be used in surgical procedures on the left hip or the right hip.

It has been found that it can be advantageous to allow the orientation of the instrument axis to be adjusted in a single plane by means of a fitting at the second end of the guide arm. Preferably the angle between that plane and an alignment axis which contains the instrument axis and the anterior superior iliac spine is at least about 15°, more preferably at least about 25°, especially at least about 30°, for example about 40 to 45°. Preferably the angle between the plane in which the instrument axis can be adjusted and the alignment axis is not more than about 65°, more preferably not more than about 55°, especially not more than about 50°. The instrument can include at least one alignment indicator which can be aligned with or with reference to the anterior superior iliac spine. Two alignment indicators can be provided on the instrument to allow the assembly to be used in surgical procedures on the left hip or the right hip. The angle between two alignment indicators can be at least about 60°, optionally at least about 70°, for example at least about 80°. The angle between two alignment indicators can be not more than about 110°, optionally not more than about 100°, for example not more than about 95°.

Optionally, the assembly can include first and second alignment indicators which extend from the instrument generally transverse to an axis defined by the instrument, with the track being positioned between the alignment indicators and the angles between the track and each of the alignment indicators being approximately equal.

Frequently, it can be preferred that the angle between the plane which contains the portion of the guide arm which extends around the patient's torso and the longitudinal axis of the patient is at least about 20°, more preferably at least about 30°, especially at least about 40°, for example about 45°. Frequently, it can be preferred that the angle between the plane defined by the portion of the guide arm and the longitudinal axis of the patient is not more than about 70°, more preferably not more than about 60°, especially not more than about 50°.

Optionally, an arcuate portion of the guide arm which extends around the patient's torso defines a path having a constant radius, and the angle of arc defined by the guide arm can be varied, for example to a maximum angle which is at least about 180°, preferably at least about 185°, for example about 190°.

Optionally, the assembly includes a fitting at or towards the second end of the guide arm, through which the instrument engages the guide arm directly or indirectly. Part of the fitting can be provided on the guide arm and part of the fitting can be provided on the instrument.

The fitting can define an orientation for an axis defined by the instrument which is fixed relative to a line extending between the first and second ends of the guide arm. The axis which is defined by the instrument will generally be an axis on which a procedure (for example a shaping procedure or an implantation of a prosthesis component) is performed using the instrument. The fitting can be arranged to allow the orientation of the instrument or its position or both to be adjusted relative to the reference axis which extends between the first and second ends of the guide arm. This can be used to adjust the angle of the instrument axis on which the acetabulum is modified using the instrument, for example to take account of factors such as particular anatomical features of the patient, or surgeon preference.

Optionally, a fitting at or towards the second end of the guide arm can allow the instrument to be moved relative to the second end of the guide arm. The fitting on the guide arm which allows the instrument to be moved relative to the guide arm can include a lock or other feature which can restrict that movement.

Optionally, the fitting can define an arcuate path for movement of the instrument relative to the second end of the guide arm. An example of a fitting can comprise a track which is fixed relative to one of the second end of the guide arm and the instrument, and a slider which is fixed relative to the other of the second end of the guide arm and the instrument and which can slide in the arcuate track. The track can be arcuate so that the instrument can move relative to the second end of the guide arm through an arc. The movement of the instrument relative to the second end of the guide arm can be along a circular path. The centre of the circle for movement of the instrument can be at or close to the centre of the acetabulum. It can be preferred that an assembly in which the fitting allows movement of the instrument through an arc includes a scale which can provide the user with an indication of the angle between an axis defined by the instrument and a line extending between the first and second ends of the guide arm. The scale can be provided on the track. The line extending between the first and second ends of the arm can provide an indication to the surgeon of a reference axis extending between a selected landmark on the patient's back features on the patient's pelvis such as the patient's acetabulum. The scale can then provide an indication to the surgeon of an angular offset of the instrument relative to the reference axis. As discussed above, one or more alignment indicators can be used to orientate the plane in which the instrument can move relative to the second end of the guide arm. The fitting can include a lock which can prevent movement of the slider relative to the track. For example, the track can be provided by two parallel plates, and the slider can include a threaded shaft which extends between the plates. A lock nut can be provided on the threaded shaft which can be tightened down against the plates.

The fitting might include a toothed rack on one part of the fitting with a resiliently deformable nib on another part of the fitting which can be slid over the teeth of the rack, with the teeth defining an array of discrete positions of the nib relative to the rack, and therefore of one of the fitting parts relative to the other fitting part.

The fitting can position the instrument so that the instrument axis (which is the axis on which the acetabulum is modified using the instrument) extends generally from the fitting at the second end of the guide arm to the mount at the first end of the guide arm. The instrument axis might extend from the fitting at the second end towards a point which is displaced from the mount at the first end of the guide arm, as might be the case for example when the guide arm has a telescoping arcuate portion and the length of the arm is adjusted telescopically to provide an adjustment of the version angle. The fitting can restrict movement of the instrument relative to the guide arm to movement along the axis which is defined by the guide arm.

A fitting at or towards the second end of the guide arm can be arranged to allow the instrument to slide in and out, towards and away from the patient's acetabulum when the instrument is in use. This can be accomplished using a fitting which includes a collar and an elongate shaft, in which one of the collar and the elongate shaft is provided at or towards the second end of the guide arm, and the instrument includes or is mounted directly or indirectly on the other of the collar and the elongate shaft. The shaft is able to slide relative to the shaft. Frequently, the shaft will be arranged so that it is directed along the axis which extends between the first and second ends of the guide arm.

It can be preferred that a fitting at or towards the second end of the guide arm can allow the orientation or the position or each of the orientation and the position of the instrument relative to the guide arm axis (extending between the first and second ends of the guide arm) to be adjusted. The instrument axis (for example on which an acetabular cup is shaped, or the cup component is implanted) can coincide with the guide arm axis. However, the possibility of adjusting the orientation of the instrument relative to the guide arm axis allows a surgeon to adjust the alignment of the instrument axis relative to the guide arm axis if desired to suit the requirements of a particular procedure. Such adjustment might be relied on by the surgeon instead of or in addition to another aspect of adjustment. For example, the surgeon might rely on adjustment of the orientation of the instrument by making an adjustment at the fitting in order to adjust the version angle instead of or in addition to adjustment of the length of an arcuate guide arm. Adjustment of the orientation of the instrument by an adjustment at the fitting can be particularly useful when the guide arm is not capable of being adjusted telescopically.

The fitting can provide a jaw which can engage the instrument in an orientation which is fixed relative the fitting. The jaw can be open on one side to allow the instrument to be positioned within the jaw. The jaw can have internal surfaces which can engage the instrument at spaced apart points along the length of the instrument and at spaced apart points around the periphery of the instrument. Preferably, the configuration of the internal surfaces of the jaw should be such that the instrument contacts the internal surfaces at three or more spaced apart points. When the instrument is in contact with the internal surfaces of the jaw at three (or more) points, the instrument should be in a stable position relative to the jaw. Preferably the jaw comprises two jaw members which can contact the instrument at spaced apart points along the instrument. Each of the jaw members can contact the instrument at spaced apart points around the periphery of the instrument. Preferably, each of the jaw members is open to one side, allowing the instrument to be slid into and out of one or both of the jaw members. The internal surfaces of the jaw members can be generally U-shaped or generally V-shaped when viewed along the length of the jaw.

The instrument can have a shaft which is engaged by a fitting at the second end of the guide arm. The shaft can have a circular cross-section so that it can rotate relative to the fitting.

The assembly can include an orientation feature at or towards the second end of the guide arm. The orientation feature can be used to orientate the guide arm rotationally around the guide arm axis (extending between the first and second ends of the guide arm). This can help the surgeon when orientating the instrument, especially when the orientation of the instrument is offset relative to the guide arm axis. An example of an orientation feature is an orientation arm which is fastened to the guide arm, generally at or towards the second end of the guide arm. The orientation arm can be fastened directly to the guide arm or indirectly (for example to a fitting on the guide arm). An orientation arm can be positioned so that, when the guide arm is arranged appropriately, the orientation arm is directed towards an appropriate anatomical landmark. For example, it can be appropriate for an orientation arm to be arranged so as to be directed towards the anterior superior iliac spine (ASIS) (especially on the same side of the patient as the acetabulum on which a procedure is being performed) to provide a reference orientation for the guide arm. This has the advantages of being easily recognised and located on many patients, and of providing a good level of accuracy when orientating the guide arm. When the assembly has been orientated using an orientation feature, an adjustment of the angle between the guide axis and the instrument axis can result in an adjustment of the anteversion angle or of the inclination angle, or of both angles with a predetermined relationship between the adjustment of the two angles.

It is envisaged that the instrument could be fastened rigidly to the guide arm so that the guide arm is part of the instrument. This arrangement would have the advantage of providing a high level of control over the orientation of the instrument axis relative to the axis which is defined by the guide arm.

The instrument can be a cutting tool. An example of a cutting tool which can be used in the assembly is a reamer which includes a cutting head having a part-spherical body with cutting teeth provided on its external surface. It can be used to modify the acetabulum by rotating it about its axis, causing the cutting teeth to act against the surface of the bone within the acetabulum. The cutting head can be manipulated manually to cut the acetabulum. It can be manipulated using a powered drive unit. The reamer will generally be mounted on a shaft with the shaft engaged by the fitting at the second end of the guide arm.

Another example of a cutting tool is a drill.

The instrument can be an inserter for an acetabular cup component. The instrument can be an impactor through which an impaction force can be applied to the acetabular cup component.

The invention also provides an instrument assembly for use in hip joint replacement surgery, which comprises:

a guide assembly comprising a reference part having a mount for locating the reference part relative to a landmark on the patient's back, and a guide part, in which the reference part and the guide part define a reference axis extending between them, and

an instrument for modifying a patient's acetabulum, which defines an axis extending through a patient's acetabulum when the instrument is in use and which is mounted on the guide part directly or indirectly,

in which the instrument can be moved relative to the guide part to adjust the instrument axis relative to the reference axis.

Optional features of the instrument assembly of the invention discussed above can be used in this instrument assembly. Examples are set out below.

Optionally, the reference part and the guide part are provided at or towards opposite ends of a guide arm which is arched to accommodate the patient's torso between the reference part and the guide part.

Optionally, the instrument assembly includes a fitting through which the instrument engages the guide part directly or indirectly, and which allows the orientation of the instrument relative to the reference axis to be adjusted.

Optionally, the fitting defines an arcuate path for movement of the instrument relative to the guide part.

Optionally, the fitting includes a track which is fixed relative to one of the second end of the guide arm and the instrument, and a slider which is fixed relative to the other of the second end of the guide arm and the instrument and which can slide in the arcuate track.

Optionally, the instrument assembly includes first and second alignment indicators which extend from the instrument generally transverse to an axis defined by the instrument, with the track being positioned between the alignment indicators and the angles between the track and each of the alignment indicators being approximately equal.

Optionally, the mount can have features which are discussed above.

The invention also provides an instrument assembly for use in hip joint replacement surgery, which comprises:

a guide assembly comprising a reference part having a mount for locating the reference part relative to a landmark on the patient's back, and a guide part, in which the reference part and the guide part define a reference axis extending between them, and

an instrument for modifying a patient's acetabulum, which defines an axis extending through the acetabulum when the instrument is in use and which is mounted on the guide part directly or indirectly,

in which the reference part and the guide part are provided at or towards opposite ends of a guide arm which is arched to accommodate the patient's torso between the reference part and the guide part, and the length of the guide arm can be adjusted to cause the orientation of the instrument relative to the guide part to change.

Optional features of the instrument assembly of the invention discussed above can be used in this instrument assembly. Examples are set out below.

Optionally, the mount can have features which are discussed above.

Optionally, the guide arm can have features such as being telescopic or arcuate or both, as discussed above.

The invention also provides instruments for use in hip joint replacement surgery which are referred to above as components of instrument assemblies. Accordingly, the invention provides an instrument for use in hip joint replacement surgery, which comprises a guide arm having first and second ends, with a mount at its first end for locating the guide arm relative to a landmark on the patient's back, and a fitting at or towards the second end of the guide arm for an instrument which can be used to modify a patient's acetabulum, in which the guide arm is arched to accommodate the patient's torso between its first and second ends, and defines a reference axis extending between its first and second ends relative to which the instrument is orientated.

The invention also provides an instrument for use in hip joint replacement surgery, which comprises a guide assembly comprising:

a. a reference part having a mount for locating the reference part relative to a landmark on the patient's back, and a guide part, in which the reference part and the guide part define a reference axis extending between them, and

b. a fitting for engaging an instrument which can be used to modify a patient's acetabulum and which defines an axis extending through a patient's acetabulum when the instrument is in use and is mounted on the guide part directly or indirectly,

in which the fitting allows the instrument to be moved relative to the guide part to adjust the instrument axis relative to the reference axis.

The invention also provides an instrument for use in hip joint replacement surgery, which comprises a guide assembly comprising:

a. a reference part having a mount for locating the reference part relative to a landmark on the patient's back, and

b. a guide part which includes a fitting for engaging an instrument which can be used to modify a patient's acetabulum, in which the reference part and the guide part define a reference axis extending between them, and

in which the reference part and the guide part are provided at or towards opposite ends of a guide arm which is arched to accommodate the patient's torso between the reference part and the guide part, and the length of the guide arm can be adjusted to cause the orientation of the instrument relative to the guide part to change.

An instrument which is provided without other components of the instrument assembly can have any of the features of the instruments which are discussed above in the context of instrument assemblies.

The invention also provides a surgical method for performance on a patient's acetabulum in hip replacement surgery, which comprises:

a. locating a bony landmark on the patient's back,

b. locating a reference point on the patient's back with reference to the bony landmark,

c. locating a reference axis which extends through a patient's acetabulum to the reference point,

d. modifying the acetabulum using an instrument which is aligned with or with reference to the reference axis.

An example of a bony landmark which is located in the first locating step is a posterior superior iliac spine. It can be preferred that the first locating step involves locating both of the posterior superior iliac spines and that the surgical method involves locating the reference point midway between the posterior superior iliac spines. The method can include incorporating an offset for the reference point relative to the bony landmark (or bony landmarks). The distance or the direction or each of the distance and direction of the offset can be varied. Factors which might affect these variables might include the patient's sex and ethnicity, and the size and other anatomical features of the patient's pelvis, the design of the cup component which is to be implanted into the patient's acetabulum, and surgeon preference.

A surgical instrument can be positioned on the skin on the patient's back at the reference point which can then be used to define the reference axis. The step of modifying the acetabulum can involve using a cutting instrument. The step of modifying the acetabulum can involve implanting a cup component in the patient's acetabulum. The modification step might be performed with the instrument aligned with the reference axis. The modification step might be performed with the instrument offset from the reference axis.

The reference axis can be located using a guide arm having a first end which is positioned at the reference point. The method can involve positioning a mount on the skin on the patient's back at the reference point, for example using an adhesive material. The guide arm can be fastened to the mount.

The method can include a step of adjusting the orientation of the instrument relative to the reference axis. The method can be performed using an assembly which includes a fitting allowing the orientation of the instrument to be adjusted. The adjustment can involve moving the instrument relative to the reference axis through an arc. The movement of the instrument relative to the reference axis can be along a circular path. The centre of the circle for movement of the instrument can be the reference point on the patient's back. The centre of the circle for movement of the instrument can be at, or more preferably close to, the centre of the acetabulum. It can be preferred that an assembly in which the fitting allows movement of the instrument through an arc includes a scale which can provide the user with an indication of the angle between an axis defined by the instrument and the reference axis. The scale can then provide an indication to the surgeon of an angular offset of the instrument relative to the reference axis.

The method can include a step of positioning a guide instrument rotationally about the reference axis by aligning an orientation instrument on the guide instrument relative to a bony landmark. An example of an appropriate bony landmark is the anterior superior iliac spine, especially on the same side of the patient as the acetabulum on which a procedure is being performed.

The invention is described below by way of example with reference to the accompanying drawings, in which:

FIG. 1 shows the relationships between anatomical reference planes and axes in the context of a patient's pelvis, and the anteversion and inclination angles as defined in the radiographic reference system.

FIG. 2 shows the relationships between anatomical reference planes and axes in the context of a patient's pelvis, and the anteversion and inclination angles as defined in the anatomical reference system.

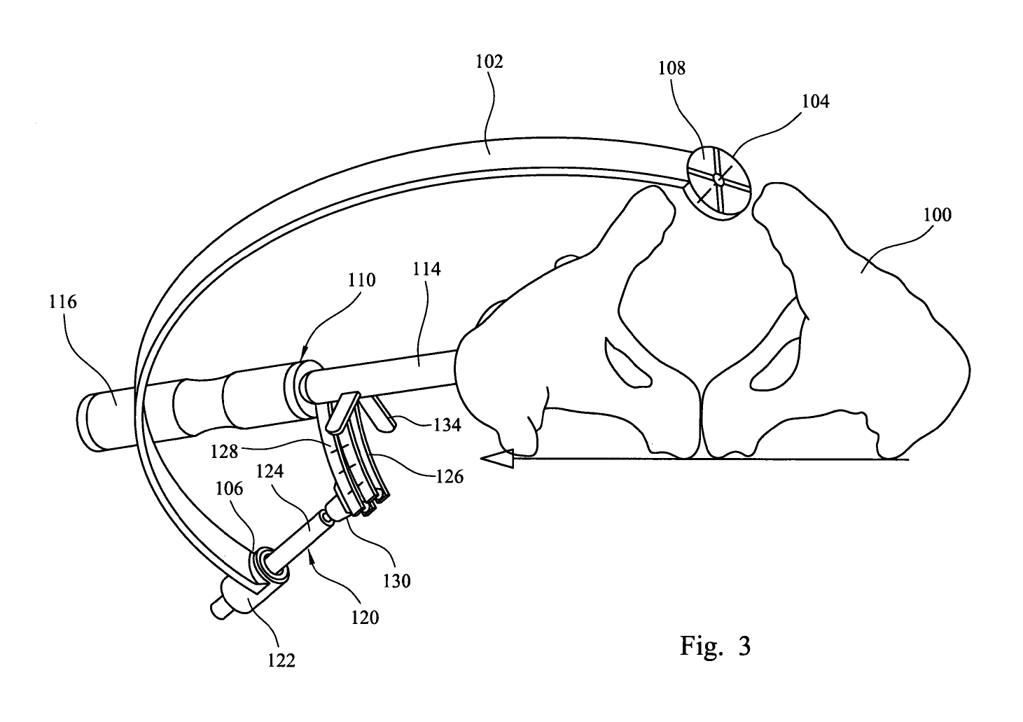

FIG. 3 is a view along the superior-inferior axis from superior to inferior of a patient's pelvis with an instrument assembly in position.

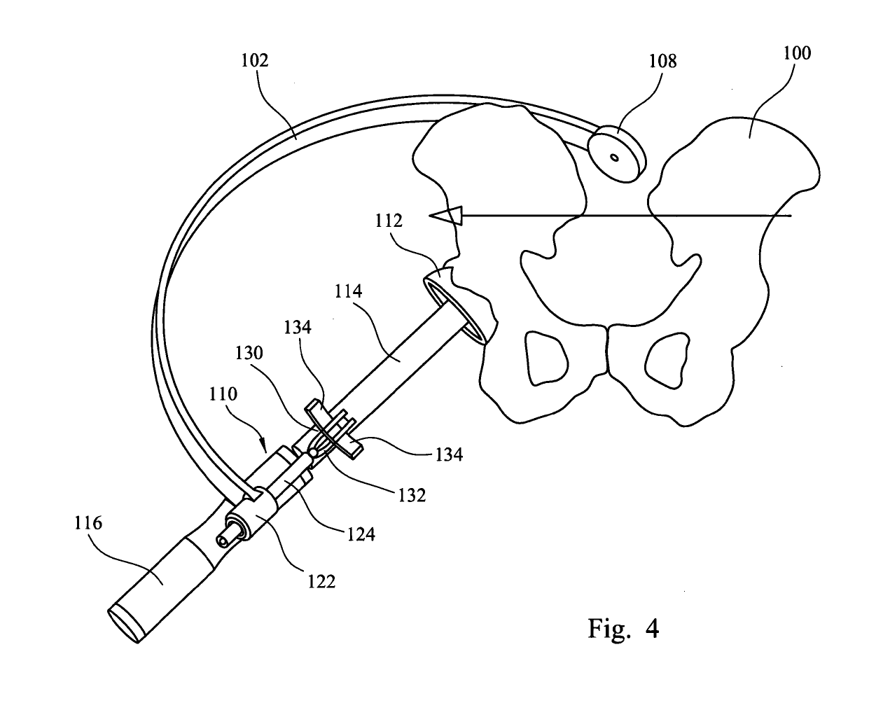

FIG. 4 is a view along the anterior-posterior axis from anterior to posterior of a patient's pelvis with the instrument assembly in position.

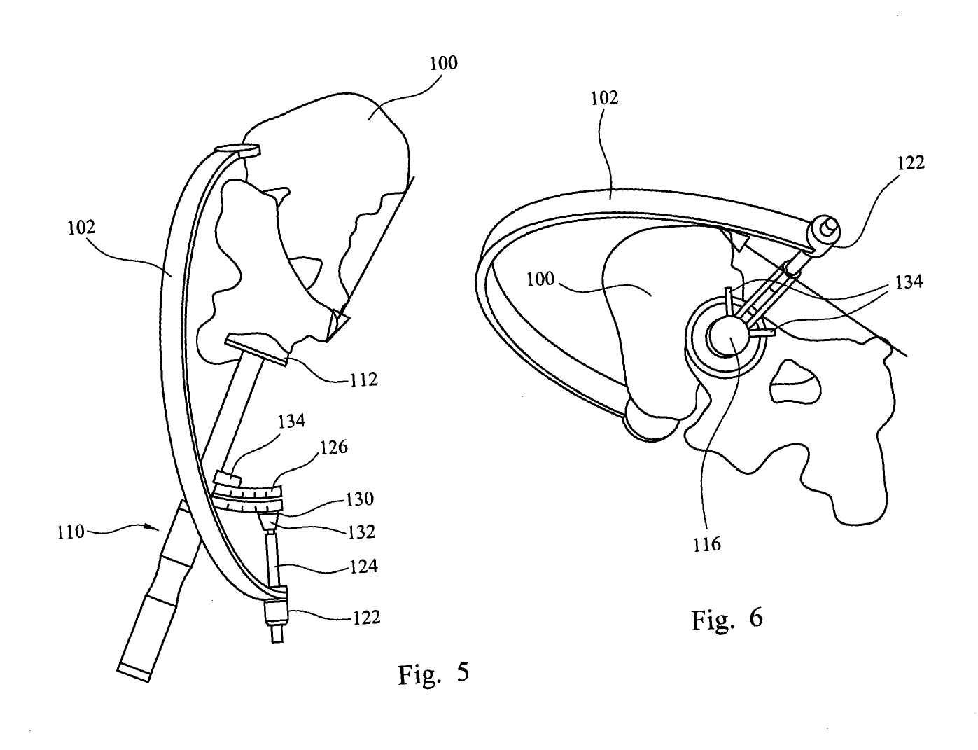

FIG. 5 is a view from a superior posterior position of a patient's pelvis with the instrument assembly in position.

FIG. 6 is a view from an inferior lateral position along the instrument which is included in the assembly for preparing the patient's acetabulum, in position within the acetabulum.

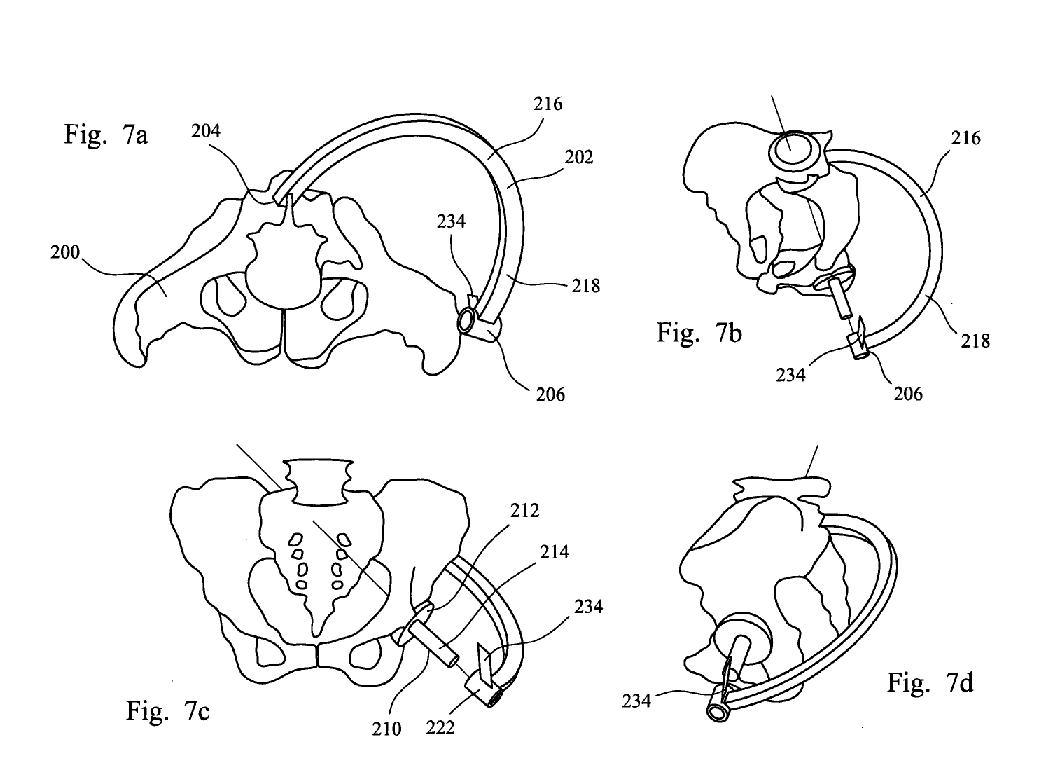

FIGS. 7a to 7d are views of a patient's pelvis with another instrument assembly in position.

Referring to the drawings, FIGS. 1 and 2 show a patient's pelvis 2 with representations of the sagittal, transverse and coronal planes 4, 6, 8 positioned on the centre of the acetabulum, and also the patient's longitudinal axis 10. FIGS. 1 and 2 also show the anatomic acetabular axis 12 which originates at the geometric centre of the acetabular socket.

FIG. 1 shows how the inclination and anteversion angles are defined in the radiographic reference system. This system relies on measurements taken from x-rays which are used for preoperative planning and used postoperatively to measure the success of the procedure. The radiographic inclination angle (0) is defined as the angle between the patient's longitudinal axis 10 and projection of the anatomic acetabular axis 12 in the coronal plane, and the radiographic anteversion angle (α) is the angle between the anatomic acetabular axis and the coronal plane.

FIG. 2 shows how the inclination and anteversion angles are defined in the anatomical reference system. This system defines the anatomical inclination angle (β) as the angle between the anatomical acetabular axis 12 and the patient's longitudinal axis 10, and the anatomical anteversion angle (γ) as the angle between the acetabular axis, as projected on to the transverse plane, and the transverse axis.

It is frequently the case for many patients that an appropriate inclination angle for an acetabular cup component using the radiographic reference system is from about 40 to about 50°, especially from about 40° to about 45°, and an appropriate anteversion angle is from about 12° to about 25°, especially from about 15 to about 25°.

The version and inclination angles that are appropriate for a surgical procedure on a particular patient can depend on several factors including the design of the component which is to be implanted in a patient, the steps in the procedure which the surgeon intends to use and, in some cases, the angle of the patient's pelvis when standing and when lying. In some cases, appropriate inclination and anteversion angles can also take account of factors such as the patient's sex and ethnicity, and the size of the patient's pelvis.

FIGS. 3 to 6 show a patient's pelvis 100 with an instrument assembly in position. The instrument assembly includes a guide arm 102 which has a first end 104 and a second end 106. The guide arm has a mount 108 at its first end.

The assembly includes an instrument which can be used to modify the acetabulum during hip replacement surgery. In the assembly shown in the drawings, the instrument is an inserter 110 which is used to position a cup component 112 in a prepared acetabulum. The instrument might be a cutting instrument such as a reamer. The inserter includes an elongate shaft 114 which has a handle 116 at one end and features (not shown) at its other end for engaging the cup component. Appropriate features on an inserter for engaging a cup component are known, for example as disclosed in WO-A-2004/069107. The exposed end face 118 of the shaft 114 can be configured so that it is appropriate for having an impaction force applied to it, for example using a hammer, as practised commonly in implantation procedures.

The assembly includes a fitting 120 at the second end of the guide arm through which the instrument 110 is connected to the guide arm. The fitting includes a collar 122 which is fastened to the guide arm at its second end. The fitting includes an elongate shaft 124 which is a sliding fit in the collar 122 so that it can translate through the collar and can rotate relative to the collar. The axis defined by the collar and the fitting shaft is directed approximately towards the first end of the guide arm.

The inserter 110 has a track 126 fastened to it which extends approximately transversely from the side wall of the shaft 114 of the inserter. The track is arcuate, being shaped as a portion of a circle whose centre is at about the centre of the acetabulum when the instrument is positioned therein. The track is formed as two plates which are spaced apart and extend approximately parallel to one another. A scale 128 is provided on at least one of the plates which indicates the extent of the angular offset from the shaft of the inserter.

A slider 130 is provided on the end of the fitting shaft 124 which slides in the collar 122 on the guide arm. The slider fits between the plates of the track 126 and can slide along the track between the plates. The slider is shaped so that cannot be pulled out from its position between the plates. The fitting shaft has an externally threaded portion towards its end, and a threaded nut 132 on the threaded portion. The nut can be advanced along the threaded portion until it contacts the edges of the plates of the track. Contact between the nut and the edges of the plates causes the slider to be clamped against sliding movement along the plates.

The inserter 110 has a pair of alignment indicators 134 fastened to it, extending from the side wall of the inserter shaft 114. The track 126 extends from the shaft between the alignment indicators.

The instrument assembly shown in FIGS. 3 to 6 can be used in a surgical procedure to modify an acetabulum, in particular (when the instrument is a cup inserter) to position a cup component in the acetabulum.

A reference point on the patient's back is located in a pre-operative planning step. The reference point is located relative to bony landmarks which can be identified by manual palpation. The posterior superior iliac spines can be used for this purpose. The reference point will usually be on the patient's superior inferior axis. The reference point might be spaced apart along the axis from the point which is midway between the posterior superior iliac spines. This can be appropriate to vary one or both of the version and inclination angles.

The reference point can be marked on the patient's skin during the planning step, for example using an ink marker. This allows the reference point to be located during the surgical procedure.

The mount 108 at the first end 104 of the guide arm 102 has a planar surface 136 which can be held against the patient's back at the reference point by a member of the surgical team. In the construction shown in the drawings, the mount is connected rigidly to the guide arm. It might be appropriate for some applications for the mount to be capable of articulation relative to the guide arm. The mount might be capable of being separated from and reconnected to the guide arm, particularly when the mount is to be fastened to the patient's skin, for example by means of a quantity of an adhesive material.

The instrument assembly is manipulated so that the fitting shaft 124 which is located within the collar 122 at the second end 106 is directed approximately towards the patient's acetabulum, and the instrument is manipulated to position the acetabular cup component 112 in the acetabulum in the patient's pelvis. Manipulation of the instrument involves sliding the fitting shaft 124 within the collar. The axis defined by the shaft 114 of the cup inserter 110, intersects the axis which extends between the first and second ends of the guide arm in the acetabulum.

The cup inserter 110 is then rotated about the axis defined by its shaft 114 until the posterior one of the two alignment indicators 134 is aligned with the anterior superior iliac spine which is on the same side of the pelvis as the acetabulum which is being modified. This arrangement can be seen in FIG. 6. Such rotation of the cup inserter involves rotation of the fitting shaft 124 within the collar 122 at the second end of the guide arm.

The angle between each of the alignment indicators 134 and the track 126 is between about 40 and 45°. This can be seen in FIG. 6. When the alignment indicator 134 is aligned with the anterior superior iliac spine, the track 126 lies approximately in the plane which is defined by the anatomical anterior-posterior axis and the axis which is defined by the shaft of the cup inserter (which coincides with the acetabular axis). The anteversion angle of the cup inserter can then be adjusted by sliding the track 126 relative to the slider 130 on the end of the fitting shaft 124. The scale 128 on the track provides an indication of the angle between the axis which extends between the first and second ends of the guide arm (represented by the fitting shaft 124) and the shaft 114 of the inserter 110.

When used in a surgical procedure on a patient with normal bone morphology, the scale 128 on the track 126 can indicate approximately the offset relative to the anteversion axis when the instrument is positioned correctly. It has been found that an appropriate anteversion angle for many patients (for example in the range 15 to 20°) is provided when the angle between the fitting shaft 124 and the shaft 114 of the inserter is about 17°. The nature of the angular adjustment of the cup inserter instrument as a result of moving the slider 130 in the track 126 can be seen in FIG. 5.

FIGS. 7a to 7d are views of a patient's pelvis 200 with another construction of instrument assembly in position. The instrument assembly includes a guide arm 202 which has a first end 204 and a second end 206. The instrument assembly includes an instrument which can be used to modify the acetabulum. The instrument is a reamer 210 which is used to shape the acetabulum to receive a cup component of a hip joint prosthesis. The reamer includes a reamer head 212 and an elongate shaft 214 which can be used to cause the reamer head to rotate. Rotational drive can be imparted to the reamer manually or using a powered drive.

The assembly includes a collar 222 at the second end of the guide arm. The reamer shaft 214 is a sliding fit in the collar which allows the reamer shaft to rotate. (In the drawings, the shaft is shown in shortened form so that it does not mask other features. In a working arrangement, the shaft will be sufficiently long to ensure that it extends through the collar.)

The guide arm has first and second arcuate portions 216, 218 between its first and second ends 204, 206. Each of the arcuate portions has the shape of an arc of a circle with a constant radius. The second arcuate portion 218 is hollow along at least part of its length with an opening where it faces towards the first end 204 of the guide arm so that the first arcuate portion 216 can be received within the second arcuate portion and can slide relative to the second arcuate portion in a telescoping manner.

The instrument has a pair of alignment indicators 234 attached to it, extending from the surface of the collar 222 at the second end 206 of the guide arm. One of the alignment indicators is visible in FIGS. 7a to 7d.

The instrument assembly can include a mount which is fastened to the skin on the patient's back at the reference point by means of an adhesive. The mount is not shown in FIGS. 7a to 7d. The mount can have at least one socket formed in it in which the first end of the guide arm can be received.

The instrument assembly shown in FIGS. 7a to 7d can be used in a surgical procedure to modify an acetabulum, in particular (when the instrument is an acetabular reamer) to shape the acetabulum to receive cup component.

A mount at the first end 204 of the guide arm 202 is positioned at the previously identified reference point.

The instrument assembly is then manipulated so that the head 212 of the reamer 210 is positioned in the acetabulum in the patient's pelvis. Manipulation of the reamer involves sliding the reamer shaft 214 within the collar 222.

The guide arm 202 is rotated about the axis which extends between its first and second ends 204, 206 so that the anterior one of the two alignment indicators 234 is aligned with the anterior superior iliac spine which is on the same side of the pelvis as the acetabulum which is being modified. This arrangement can be seen in FIG. 7d.

With the angular orientation of the guide arm set with reference to an appropriate bony landmark such as the anterior superior iliac spine, adjustment of the length of the guide arm using the telescoping features can be used to adjust the orientation of the reamer instrument relative to the anatomic acetabular axis. It can be preferred that the angle between the alignment indicator and the plane defined by the arcuate portions of the guide arm is such that the adjustment of the length of the guide arm gives rise to a variation in the anteversion angle of the reamer instrument without also changing significantly the inclination angle.

1. An instrument assembly for use in hip joint replacement surgery, which comprises a guide arm having first and second ends, with a mount at its first end for locating the guide arm relative to a landmark on the patient's back, and an instrument at or towards the second end of the guide arm for modifying a patient's acetabulum, in which the guide arm is arched to accommodate the patient's torso between its first and second ends, and defines a reference axis extending between its first and second ends relative to which the instrument is orientated.

2. An instrument assembly as claimed in claim 1, which includes a fitting at or towards the second end of the guide arm, through which the instrument engages the guide arm directly or indirectly.

3. An instrument assembly as claimed in claim 2, in which the fitting restricts movement of the instrument relative to the guide arm to movement along an axis which is defined by the instrument.

4. An instrument assembly as claimed in claim 2, in which fitting allows the orientation of the instrument relative to the reference axis to be adjusted.

5. An instrument assembly as claimed in claim 4, in which the fitting defines an arcuate path for movement of the instrument relative to the second end of the guide arm.

6. An instrument assembly as claimed in claim 5, in which the fitting includes a track which is fixed relative to one of the second end of the guide arm and the instrument, and a slider which is fixed relative to the other of the second end of the guide arm and the instrument and which can slide in the arcuate track.

7. An instrument assembly as claimed in claim 6, which includes first and second alignment indicators which extend from the instrument generally transverse to an axis defined by the instrument, with the track being positioned between the alignment indicators and the angles between the track and each of the alignment indicators being approximately equal.

8. An instrument assembly as claimed in claim 1, in which the mount includes a quantity of adhesive on a surface which contacts the patient's skin when the mount is in place.

9. An instrument assembly as claimed in claim 1, in which at least a portion of the guide arm between the first and second ends is arcuate, defining a generally rounded arc.

10. (canceled)

11. An instrument assembly as claimed in claim 9, in which the angle of arc defined by the arcuate portion of the guide arm is at least about 150°.

12. (canceled)

13. An instrument assembly as claimed in claim 1, in which the angle between surface of the mount which is intended to be fitted against the patient's back and a plane which is defined by the arcuate portion of the guide arm is at least about 120°.

14. An instrument assembly as claimed in claim 1, in which the angle between surface of the mount which is intended to be fitted against the patient's back and a plane which is defined by the arcuate portion of the arm is not more than about 170°.

15. An instrument assembly as claimed in claim 1, in which the arched portion of the guide arm is telescopic.

16. (canceled)

17. An instrument assembly as claimed in claim 1, in which the instrument is one of a cutting tool or an inserter for a cup component.

18. (canceled)

19. An instrument assembly for use in hip joint replacement surgery, which comprises:
a. a guide assembly comprising a reference part having a mount for locating the reference part relative to a landmark on the patient's back, and a guide part, in which the reference part and the guide part define a reference axis extending between them, and
b. an instrument for modifying a patient's acetabulum, which defines an axis extending through the acetabulum when the instrument is in use and which is mounted on the guide part directly or indirectly,
in which the instrument can be moved relative to the guide part to adjust the instrument axis relative to the reference axis.

20. An instrument assembly as claimed in claim 19, in which the reference part and the guide part are provided at or towards opposite ends of a guide arm which is arched to accommodate the patient's torso between the reference part and the guide part.

21. An instrument assembly as claimed in claim 19, which includes a fitting through which the instrument engages the guide part directly or indirectly, and which allows the orientation of the instrument relative to the reference axis to be adjusted.

22. An instrument assembly as claimed in claim 21, in which the fitting defines an arcuate path for movement of the instrument relative to the guide part.

23. An instrument assembly as claimed in claim 22, in which the fitting includes a track which is fixed relative to one of the second end of the guide arm and the instrument, and a slider which is fixed relative to the other of the second end of the guide arm and the instrument and which can slide in the arcuate track.

24. An instrument assembly as claimed in claim 23, which includes first and second alignment indicators which extend from the instrument generally transverse to an axis defined by the instrument, with the track being positioned between the alignment indicators and the angles between the track and each of the alignment indicators being approximately equal.

25. (canceled)

26. A surgical method for performance on a patient's acetabulum in hip replacement surgery, which comprises:
a. locating a bony landmark on the patient's back,
b. locating a reference point on the patient's back with reference to the bony landmark,
c. locating a reference axis which extends through the acetabulum to the reference point,
d. modifying the acetabulum using an instrument which is aligned with or with reference to the reference axis.

27. A surgical method as claimed in claim 26, in which the bony landmark is a posterior superior iliac spine.

28. A surgical method as claimed in claim 27, in which both of the posterior superior iliac spines are located and in which the surgical method involves locating the reference point midway between the posterior superior iliac spines.

29. A surgical method as claimed in claim 26, in which the target is positioned on the skin on the patient's back at the location which is identified with reference to the bony landmark.

30. A surgical method as claimed in claim 26, in which the step of modifying the acetabulum involves using a cutting instrument.

31. A surgical method as claimed in claim 26, in which the step of modifying the acetabulum involves implanting a cup component in the patient's acetabulum.

32. A surgical method as claimed in claim 26, which includes a step of aligning the instrument with one of the patient's anterior superior iliac spines by rotating the instrument about its axis.

33. (canceled)

34. (canceled)

35. (canceled)
